(19) United States
(12) Patent Application Publication
(10) Pub. No.: US 2010/0261179 A1
(43) Pub. Date: Oct. 14, 2010

(54) SAMPLE PREPARATION DEVICES AND ANALYZERS

(75) Inventors: Jason Betley, London (GB); Barry Boyes, London (GB); John Walter Czajka, London (GB); Michael Roy Fairs, London (GB); Colin Fewster, London (GB); Thomas David Ford, London (GB); Douglas Jason Green, London (GB); Piers Sebastian Harding, London (GB); Gary Stephen Howard, London (GB); Jay Lewington, London (GB); William Richard Mawer, London (GB); Catherine Mills, London (GB); James Onslow, London (GB); Carmelo Volpe, London (GB)

Correspondence Address: FOLEY AND LARDNER LLP;SUITE 500, 3000 K STREET NW, WASHINGTON, DC, 20007, US

(73) Assignee: Smiths Detection-Watford Ltd.

(21) Appl. No.: 12/682,123

(22) PCT Filed: Oct. 17, 2008
(86) PCT No.: PCT/US08/11910

§ 371 Date: Jun. 8, 2010

(30) Foreign Application Priority Data
Oct. 17, 2007 (GB) 0720264.1

(51) Int. Cl. C12Q 1/68; B01D 35/06; C12M 1/34

(52) U.S. Cl. 435/6; 210/222; 435/287.2

(57) ABSTRACT

The application provides sample preparation devices and analyses. The devices and analyzes allow for the rapid preparation and analysis of samples using a variety of techniques, including PCR, by even unskilled users.

CROSS-REFERENCE TO RELATED PATENT APPLICATIONS

Applicants claim priority to GBRI Priority Application 0720264.1, filed Oct. 17, 2007 including the specification, drawings, claims and abstract, which is incorporated herein by reference in its entirety.

### FIELD OF THE APPLICATION

This application relates to sample preparation devices and analyzers. The application is more particularly concerned with devices for preparing biological samples into a form suitable for subsequent analysis.

### BACKGROUND

Analysis or detection equipment is available that can reliably identify specified biological substances by means of polymerase chain reaction (PCR) amplification and fluorescence identification. The polymerase chain reaction is a well known technique for amplification of small amounts of a specific DNA sequence to produce larger amounts of that specific DNA sequence, at which point the specific products can be identified or visualised in a number of ways. A variant of the polymerase chain reaction uses RNA as its input: reverse transcription of the RNA to its complementary DNA sequence is followed, optionally in the same reaction mixture, and optionally by the same enzyme, by polymerase chain reaction amplification of the complement DNA.

This technique, although extremely powerful, is prone to inhibition by a wide variety of inhibitors, found widely in most sample types. There are well-established sample preparation methods in the prior art for addressing this issue, which rely either on the use of kits by trained molecular biologists, or the use of large laboratory robotic systems using reagents and plastic consumables. Portable PCR analysis equipment (such as the Bio-Seeq sold by Smiths Detection—Watford Limited) is available, which can be used easily with little training. Examples of preparation apparatus are described in WO05/121963, WO06/090127, WO06/079814, EP1383602, WO05/106040, WO05/019836 and GB0704035.5, which are hereby incorporated by reference in their entirety.

### SUMMARY

Provided herein are alternative sample preparation devices and analyzers.

According to a first aspect there is provided an arrangement for separating magnetic particles from a fluid including a container of the fluid and magnetic particles having a tapering, closed lower end portion, and magnet means selectively positionable relative to the container adjacent its lower end, the magnet means including two magnet pole pieces aligned substantially parallel with the axis of the container and arranged to form regions for collection of the magnetic particles along the lower end portion and displaced from the lower end of the end portion.

According to a second aspect there is provided an arrangement for separating magnetic particles from a fluid including a container of the fluid and magnetic particles, and magnet means movable between a first position where its magnetic field attracts the magnetic particles to a surface of the container and a second position where its magnetic field has substantially no effect on the magnetic particles, the magnet means being movable along curved path in a plane substantially at right angles to the axis of the container.

According to a third aspect there is provided an arrangement for dispensing a fluid into a cuvette including a capillary tube open at both ends and extending within the cuvette with the lower end of the capillary in contact with the inside of the closed end of the cuvette and with the upper end of the capillary exposed at the upper end of the cuvette, the dimensions of the capillary and cuvette being arranged such that when the fluid is applied to the upper end of the capillary it flows to its lower end expelling air in the cuvette between the outside of the capillary and the inside of the cuvette.

According to a fourth aspect there is provided a sample preparation device including an arrangement according to the above first, second or third aspects.

According to a fifth aspect there is provided a sample preparation device including a fluid-transfer mechanism arranged to transfer material between different containers in the device, the mechanism having two components movable relative to one another to define a chamber of variable volume and a dispensing device connected with the chamber, the dispensing device being displaceable such that it can be raised and lowered relative the containers, the displacement of the dispensing device and the two components relative to one another both being brought about by rotary means.

The two components preferably include a barrel and a piston. The rotary drive is preferably provided by an external unit to which the sample preparation device can be mounted and demounted.

According to a sixth aspect there is provided a sample preparation device having a sample inlet including manual macerator means by which a fluid material is derived from a liquid, solid or semi-solid sample material preparatory to further preparation.

The macerator means caninclude at least one reservoir of a treatment fluid protected by a breakable seal, the treatment fluid being brought into contact with the sample material by manual operation of the macerator means. The macerator means caninclude a rotatable macerator knob that seals the inlet and is screw threaded relative to the sample inlet such that rotation of the knob displaces it down to push the sample down through the macerator means.

According to a seventh aspect there is provided a sample preparation device having a plurality of containers of substances used in preparation of a sample, a transfer device that is arranged to be raised and lowered and moved laterally relative to the containers to transfer substances between containers, the device including an absorbent material arranged to collect excess substance at the lower end of the transfer device.

The absorbent material can be disposed on the upper surface of the containers and may be a fabric or non-woven material, for example. The sample preparation device can be arranged to lower the transfer device such that its lower end contacts the absorbent material when it is necessary to remove excess fluid from the end of the transfer device.

According to an eighth aspect there is provided an analyzer arrangement including a PCR analyzer and a sample preparation device according to any one of the fourth to seventh aspects.

According to a ninth aspect there is provided an analyzer arrangement including a base unit and a sample preparation device mountable with and demountable from the base unit, the sample preparation device including at least one container containing a mixture of a fluid and magnetic particles and the sample preparation device including means for extracting fluid from the container, the base unit including means operable to move magnet means selectively between a first position where a magnetic field from the magnet means attracts the magnetic particles to a surface of the container such that the fluid can be extracted and the magnetic particles retained in the container and a second position where the magnetic filed has substantially no effect on the magnetic particles.

The magnet means can be movable along an arc in a plane at right angles to the axis of the container, and can be mounted on the sample preparation device.

According to a tenth aspect there is provided an analyzer assembly including an analyzer and a sample preparation device mountable with and demountable from the analyzer, the sample preparation device including a plurality containers of substances for preparing the sample, some at least of the containers being provided together as a single component and at least one of the containers being provided separately of the component and being mountable with the component, the separately-provided container being provided with a machine-readable identification that can be read by the analyzer to control the analyzer to drive the sample preparation device in a particular sequence.

The component providing at least some of the containers may be a circular carousel, which may be rotatable relative to the sample preparation device.

According to an eleventh aspect there is provided a method of identifying a sample substance, including providing a sample preparation device having a plurality of installed containers of substances suitable for use in preparation of a range of different substances, providing a range of at least two separate containers containing different substances suitable for use in preparation of at least two respective different substances, selecting one of the separate containers according to the substance to be detected, installing the selected separate container with the sample preparation device, adding the sample substance to the sample preparation device and operating the sample preparation device to prepare the substance.

According to a twelfth aspect there is provided apparatus for carrying out a method according to the above eleventh aspect.

### BRIEF DESCRIPTION OF THE DRAWINGS

A portable sample analysis device assembly including an analyzer and a sample preparation device and its method of operation will now be described, by way of example, with reference to the accompanying drawings, in which:

FIG. 1 is a perspective view of an exemplary analyzer assembly;

FIG. 2 is a perspective view of an exemplary sample preparation device (2) from a side;

FIG. 3 is a perspective view of the exemplary sample preparation device of FIG. 2 from the opposite side of that shown in FIG. 2;

FIG. 4 is a side elevation view of the exemplary sample preparation device of FIGS. 2 and 3 from one side;

FIG. 5 is an end elevation view of the exemplary sample preparation device of FIGS. 2 and 3;

FIG. 6 is an enlarged cross-sectional side elevation view of a lower part of the exemplary sample preparation device of FIGS. 2 and 3 showing a part of the drive mechanism;

FIG. 7 is a view of the underside of the exemplary sample preparation device of FIGS. 2 and 3;

FIG. 8 is an enlarged excerpt of FIG. 7 ;

FIG. 9 is an enlarged cross-sectional side elevation view of a lower part of the sample preparation device of FIGS. 2 and 3 showing a part of the cuvette filling arrangement;

FIG. 10 is an exploded view of the sample inlet port of FIGS. 2 and 3;

FIG. 11 is a perspective view of the interior of the sample preparation device of FIGS. 2 and 3, with its housing removed and with the pipette in a raised position;

FIG. 12 is a cross-sectional, side elevation view of the interior of the sample preparation device of FIGS. 2 and 3 with the pipette in a lowered position for filling the cuvette;

FIG. 13 is a perspective view of the outside of the sample preparation device of FIGS. 2 and 3 showing the macerator knob and the reagent cartridge prior to insertion;

FIGS. 14, 15 and 16 are cross-sectional side elevation views of the sample preparation device of FIGS. 2 and 3 showing a macerator knob being inserted;

FIGS. 17 and 18 are cross-sectional side elevation views of the sample preparation device of FIGS. 2 and 3 showing stages in filling of a cuvette;

FIG. 19 is a plan view of a mounting bay of an exemplary analyzer instrument;

FIG. 20 is a sectional elevation view showing an interaction of a magnet assembly with a container of the carousel;

FIG. 21 is an exploded perspective view of an exemplary reagent cartridge (27) as shown in FIGS. 2 and 3:

FIG. 22 is a plan view of an exemplary carousel of a sample preparation device;

FIG. 23 is a perspective view of the carousel of FIG. 22;

FIG. 24 is a cross-sectional side elevation view of an exemplary cuvette filling mechanism; and

FIG. 25 is a perspective view showing how a pipette is mounted.

### DETAILED DESCRIPTION

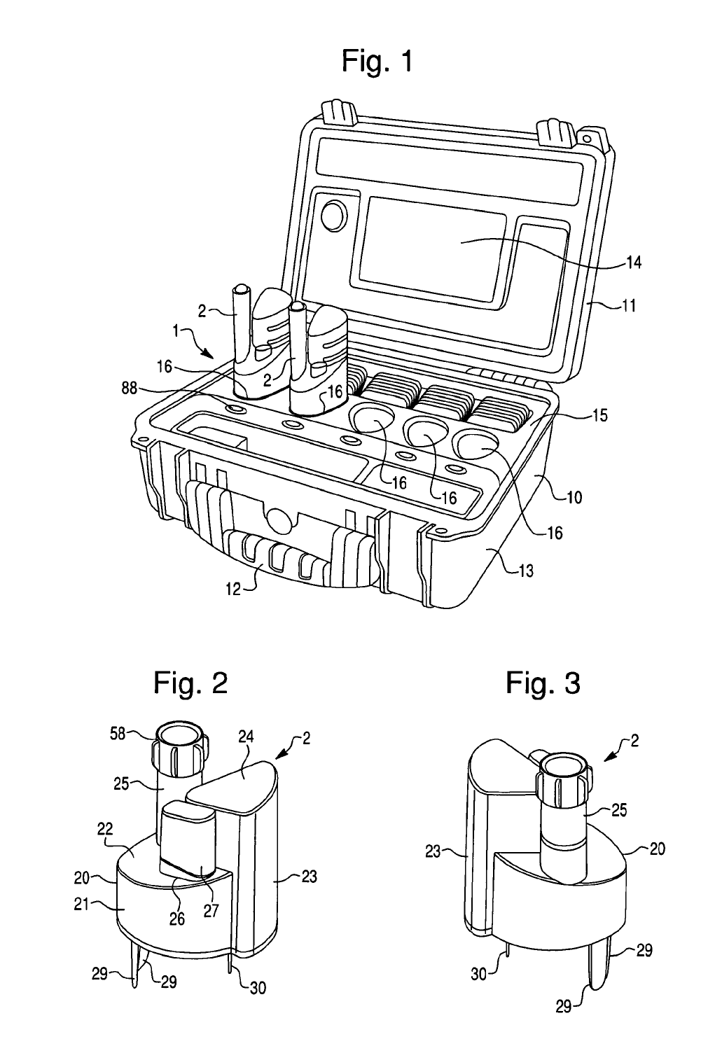

With reference to FIG. 1, an assembly can comprise a combination of an analyzer or base unit 1, by which analysis is carried out, and a sample preparation device 2 by which samples taken from the field can be prepared into a form suitable for analysis by the analyzer. The analyzer 1 can, optionally, include a rugged outer case 10 with a hinged lid 11 and a carrying handle 12. The base 13 of the case 10 contains a PCR analysis instrument or other instrument capable of one or more suitable analysis methods. The instrument may use any suitable nucleic acid amplification method, such as, for example, conventional PCR or Linear After the Exponential PCR as described in, for example, U.S. Pat. No. 7,198,897. Thermocycling may be carried out using any acceptable method, such as, for example, conventional heating and cooling elements or thermoelectric elements. Detection may be achieved using any suitable method, such as fluorimetric methods. In one embodiment fiberoptic fluorimetry can be used. Conventional PCR analyzers are known, such as, for example, the Bio-Seeq analyzer available from Smiths Detection. The instrument may also use any suitable analytical techniques or a combination of analytical techniques, including mass spectrometry, gas spectrometry, ion mobility spectrometry, and antibody binding methods, for example. In one embodiment, the lid 11 of the case can support, on its inside surface, a display screen 14, such as an LCD screen or an touch screen. The LCD touch screen can be used to input instructions to control the device or to display information, such as the results of analysis or system parameters. The sample analysis device can include any number of mounting bays each adapted to receive a sample preparation device. In one embodiment, the upper surface 15 of the analyzer base 13 can have any number of mounting bays 16. In another embodiment, the upper surface 15 of the analyzer can have five mounting bays 16, arranged in any configuration, such as, for example a row, to which five different sample preparation devices 2 (only two shown fitted in FIG. 1) can be removably fitted. In another embodiment, the analyzer can have auxiliary, for example, slave, mounting bays that can communicate with the master analyzer. It will be appreciated that the analyzer could have any number of one or more bays 16 to which sample preparation devices 2 are fitted.

In one embodiment, the analyzer 1 can be sealed against ingress of fluids, and all external surfaces can be resistant to normal cleaning fluids. In a further embodiment, the analyzer is substantially completely sealed against ingress of fluids. This enables the analyzer to be immersed in a cleaning fluid, with the case 10 open or closed, to ensure that any harmful substances on the analyzer are rendered harmless. The analyzer 1 is capable of carrying out sample analysis, such as PCR analysis, on each of the samples prepared by sample preparation devices 2 at the same or at different times. This enables the sample preparation devices 2 to be fitted to the analyzer 1 as and when they become available and for the sample preparation stages for each sample to be started immediately. The devices can be fitted so that the analysis can take place as soon as the samples have been appropriately prepared. In one embodiment, the analyzer 1 can provide all the motive power for the sample preparation devices 2 through appropriate mechanical couplings, for example, to be described later, so that the sample preparation devices do not themselves need to include any motor or battery. This helps to keep the cost, size and weight of the sample preparation devices 2 to a minimum and reduces disposal problems, volume, and cost.

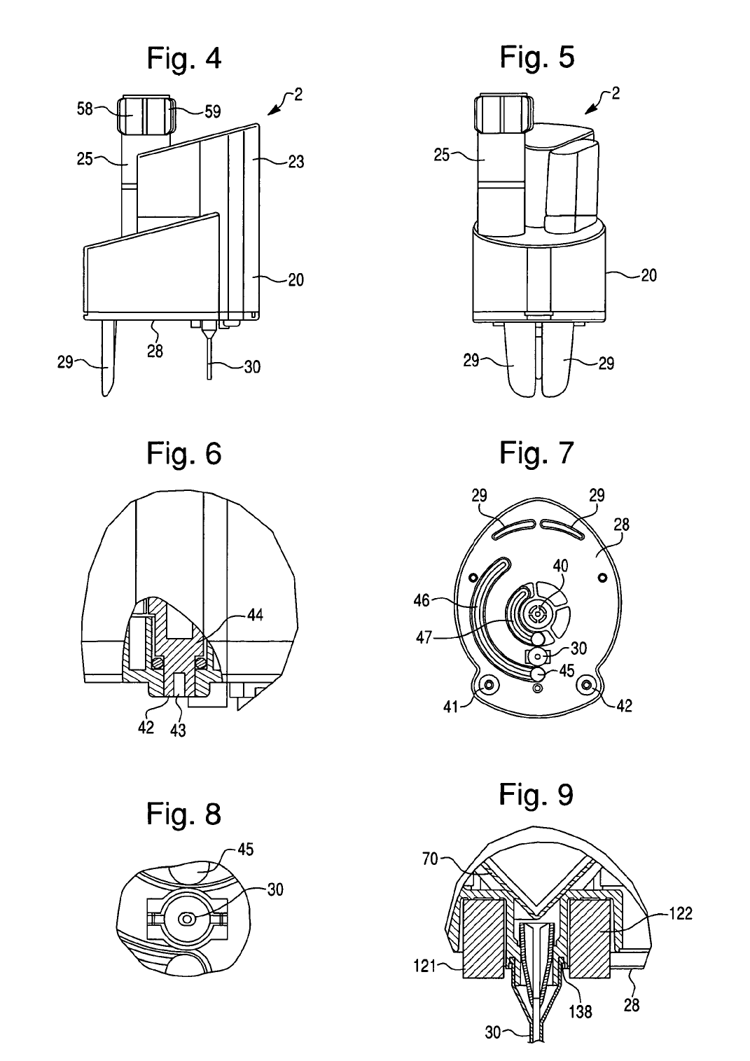

With reference now also to FIGS. 2 to 9, an exemplary sample preparation device 2 will be described in greater detail. The device 2 can include an outer housing 20 of any suitable material and shape. In one exemplary embodiment, the outer housing can be a molded plastic outer housing 20 of generally oval section. In this exemplary embodiment, the housing 20 has a base section 21 with an inclined upper surface 22, a substantially triangular, wedge-shape fluid transfer enclosure 23 extending to about twice the height of the base section and having a upper surface 24 inclined at a slightly shallower angle than that on the base. The height of the enclosure 23 can be any suitable height. In one embodiment, the enclosure height can be about 100 mm. The housing 20 also can have a inlet cylinder 25 extending approximately vertically up to the same level as the top of the upper surface 24. The upper surface 22 of the base section 21 is interrupted by an elongated slot 26 extending along side one side of the enclosure 23. This slot 26 can be used to fit a reagent cartridge 27, to be described in more detail later.

In the exemplary embodiment shown in FIGS. 2-9, on the underside 28 of the housing 20 are mounted two rigid alignment tongues 29, which project substantially vertically down. In one embodiment, the alignment tongues can be from approximately 10 mm to approximately 50 mm and in a further embodiment the alignment tongues can be approximately 39 mm. The tongues 29 can be arranged in any suitable arrangement and can be rounded at their lower ends 30 and can be closely spaced from one another. The tongues 29 can be shaped and positioned to align with alignment apertures 129 (FIG. 19) located in the bays 16 on the analyzer 1 to ensure correct alignment of the preparation devices 2 with respect to the bays. The length of the tongues 29 also ensures that the preparation devices 2 can only be loaded on the analyzer 1 when oriented substantially vertically with respect to the upper surface 15 of the analyzer. Also projecting substantially vertically downwards from the underside 28 of the device 2 is a cuvette 30 into which the prepared sample is dispensed for analysis. The cuvette 30 can be quite delicate and the alignment tongues 29 ensure that it is correctly aligned with a reception aperture 130 (FIG. 19) in the bay 16 during loading of the device 2 on the analyzer. When the cuvette 30 is loaded in the reception aperture 130, it extends into, for example, a PCR analysis module for that bay so that material in the cuvette is subject to PCR analysis. The cuvette 30 will be described in greater detail later. During storage and before use of the sample preparation device 2, a removable cap 31 (FIG. 13) can be fitted over its lower end to protect the cuvette 30. This cap 31 can be removed just before loading the device into the mounting bay 16.

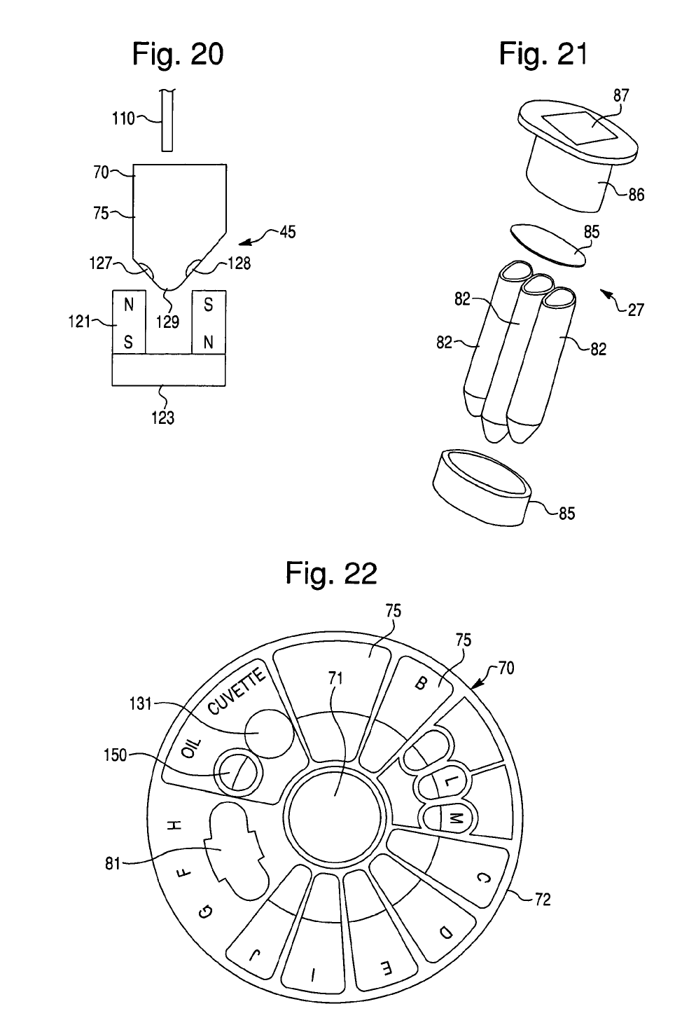

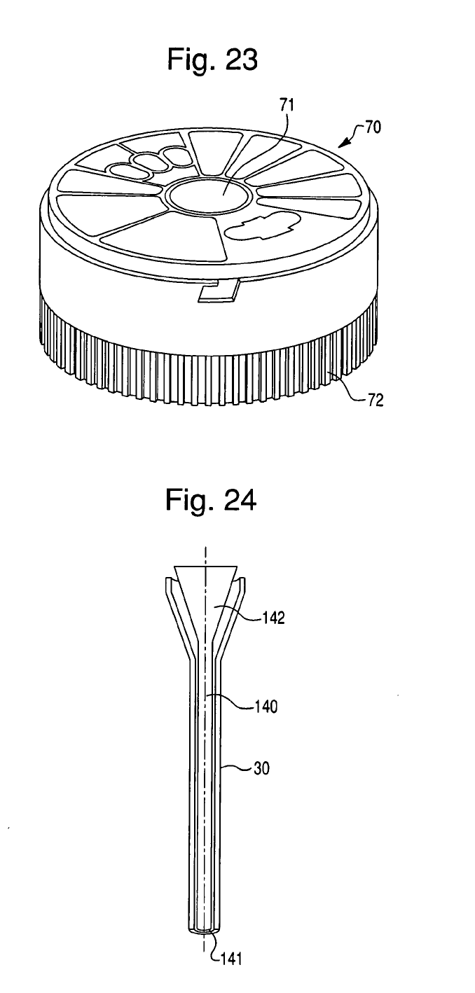

In an exemplary embodiment, the underside 28 of the housing 20 also can include three mechanical, rotary drive input couplings 40, 41 and 42. One coupling 40 can be located centrally and the other two 41 and 42 can be located close to the edge, in respective corners of the enclosure 23. The input couplings 40 to 42 each can take the form shown in FIG. 6 of a tapered socket 43 of square section in the end of a vertically-oriented drive shaft 44. Each socket 43 is adapted to receive a correspondingly-shaped male head of a respective drive element 140 to 142 (FIG. 19) located in each bay 16 on the analyzer 1 and mechanically connected to respective motors (not shown) in the analyzer. A magnet assembly 45 (shown in more detail in FIG. 20), which can be movable in, for example, a substantially horizontal plane along two parallel slots 46 and 47, also can be provided on the underside 28 of the housing 20. The slots 46 and 47 can be arcs curved in, for example, a part-circular shape with a common radius centered on the central drive coupling 40.

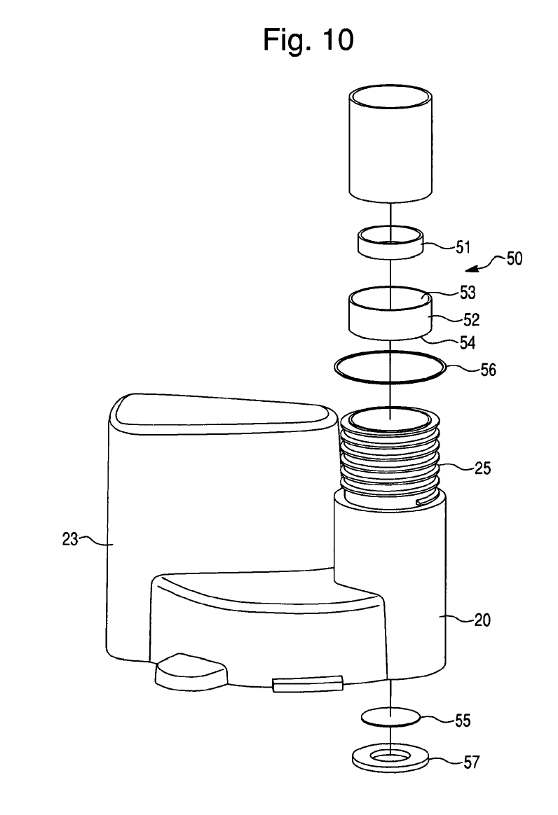

The internal features of an exemplary preparation device 2 will now be described with reference to FIGS. 10 to 18.

An exemplary sample inlet 25 is shown in more detail in FIGS. 10, 14, 15 and 16 and includes within it a sample homogenization module 50 of cylindrical shape and containing a displaceable macerator plate 51 of an open construction above a reservoir 52 containing a lysis/binding buffer with breakable seals 53 and 54 (such as of a foil) on its upper and lower surface. The module 50 can be positioned above filter 55 held in place between O-ring seals 56 and 57. The filter 55 can be made of any suitable material, including polypropylene, and can be coarse. The inlet 25 can be completed by a macerator knob 58. In one embodiment, the macerator knob can have a surface configured to ease hand turning, such as a friction-enhancing surface or a knurled outer surface 59. The macerator knob also can include a screw-threaded inner surface 60, which engages a screw thread 61 on the outside of the inlet 25. The knob 58 can have a coaxial plunger 62 on its inside, which makes a close sliding fit in the bore of the inlet 25. The knob 58 can be removed to insert the sample and is then screwed down to effect maceration.

The sample may be any suitable sample, including, but not limited to any bodily fluids, blood, sputum (respiratory tract secretions/scraping), milk, feces; solid unknowns, including powders (such as anthrax spores), soft tissue (skin, muscle, hair follicle, vesicle), vegetable materials, and soil. Other sample substances or mixtures of substances also are possible.

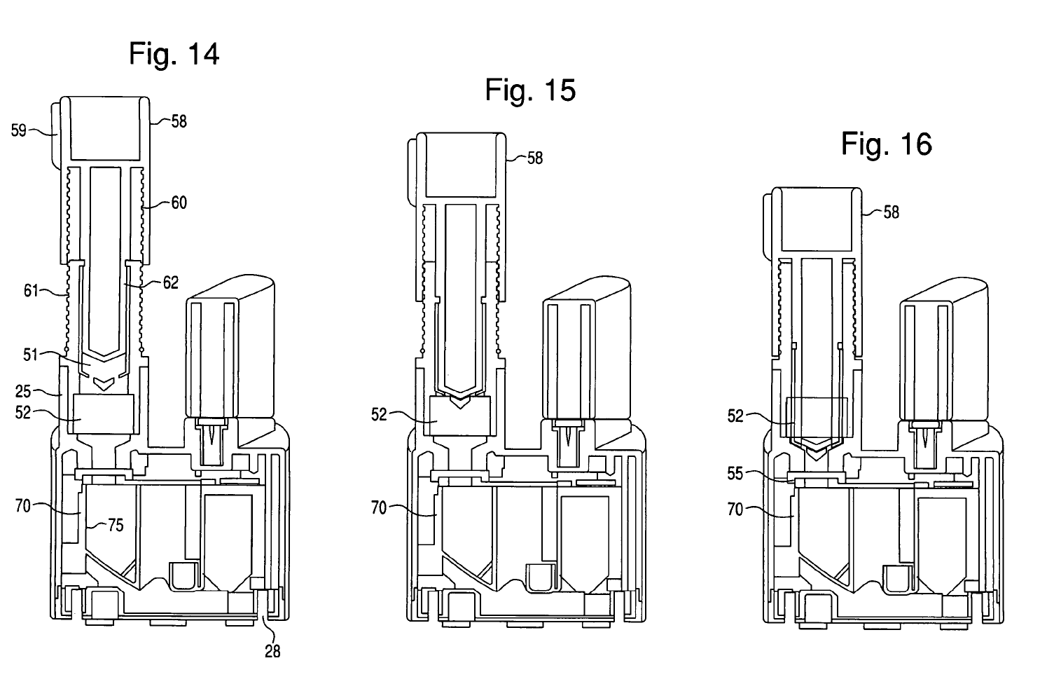

After the sample has been placed in the inlet 25, the knob 58 can be replaced and manually screwed down so that its plunger 62 is also moved down to engage and move down the macerator plate 51, as shown in FIG. 14. The plate 51 pushes through and breaks the seal 52 on the upper surface of the buffer reservoir 50, which can hold, for example, lysis/binding buffer, so that the sample is exposed to this liquid, as shown in FIG. 15. The lysis/binding buffer releases nucleic acids from the sample and inactivates nucleases so that the nucleic acids come out into solution. The lysis and nuclease inactivation steps can be performed together or separately. In one embodiment, the lysis and nuclease inactivation steps are performed at approximately the same time. In this exemplary embodiment, further rotation of the macerator knob 58 causes its plunger 62 to break through the lower seal on the reservoir 50 and pushes the mixture of the sample, lysis and binding buffer down through the filter 55, as shown in FIG. 16.

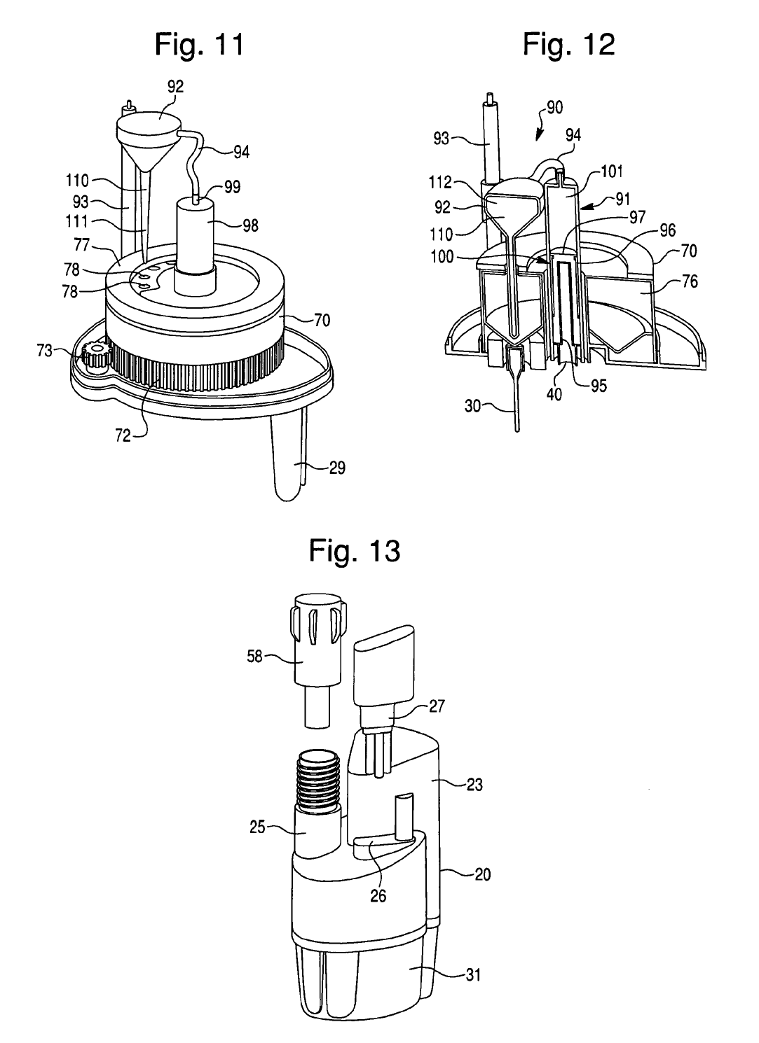

The mixture can drop by gravity from the filter 55 into the first container or pot 75 in a rotatable carousel 70, labeled “A” in FIG. 22. In an embodiment, the carousel 70 can be molded of a plastics material having a circular shape with a central, circular aperture 71. A toothed rack 72 extends around the outside of the carousel 70 at its lower end, which can be used to rotate the carousel about its axis. The rack 72 is engaged by, for example, a pinion wheel 73 (FIG. 11) mounted at the edge of the base of the housing 20 and which is rotated by its input drive coupling 41 when engaged by the drive coupling 141 on the analyzer 1. Internally, the carousel 70 is divided into wedge shape recesses (also referred to herein as pots or containers) 75 of varying sizes, most of which provide containers or pots for use in the various treatment stages to which the sample is subjected. In one embodiment, the carousel is divided into five to 20 pots, and in a further embodiment, the carousel is divided in to 6, 7, 8, 9, 10, 11, 12, 13, 14, 15, 16, 17, 19, or 19 pots. The pots 75 can contain various substances with which the sample can be mixed to produce a product prepared in a form suitable for analysis. Some of the pots 75 can be left empty for mixing purposes or containing waste material. In general, the pots 75 may contain various of the following: buffers of various pH and composition; enzymes (including nucleic acid modifying enzymes of various kinds in aqueous or freeze-dried form); deoxynucleotides; metal ions; oligonucleotides (including those labelled with reporter molecules such as fluorophors); and proteins. All the pots 75 containing a reagent or treatment substance can be covered before use by a pierceable covering 76, such as, for example, a foil covering, to prevent escape of the contents. On top of the covering 76 there can be an upper cover having an exposed upper surface layer 77 of a fluid-absorbing material, such as a wadding of paper, fabric, woven or non-woven or a coating of absorbent chemical. The upper cover can have an impermeable base of a plastics material, not shown. The purpose of this will become apparent later. The upper cover and layer 77 is cut with small openings 78 to expose the underlying cover 76 in alignment with the pipette 110. The first pot “A” is relatively large and is empty before it receives the sample mixture. By way of example, for a PCR analysis reaction, the pots labeled “B”, “C”, “D”, “E”, “I”, “J”, “K”, “L” and “M” can contain the following substances:
B—empty at start
C—lysis binding buffer
D—wash buffer No 1
E—wash buffer No 2
I—wash buffer No 3
J—empty—used for waste from lysis
K—elution buffer
L—DNAse buffer
M—proteinase K and magnetic beads

According to the particular substances being prepared, it will be appreciated that different treatment substances could be used and that different numbers of pots 75 could be provided. The sample preparation device 2 can be used in a range of at least two different preparation sequences, such as for use in preparing samples for detection of different substances. Some of the treatment substances and preparation steps are common to the range and these treatment substances can be provided in the pots “B”, “C”, “D”, “E”, “I”, “J”, “K”, “L” and “M”. Others of the treatment substances vary for the different preparations and these can be provided in a cartridge 27 (FIG. 21) provided separately of the sample preparation device 2 and inserted into it prior to use.

The cartridge 27 can be shaped to extend in the elongate slot 81 extending at right angles to a radius of the carousel 70. The cartridge 27 can be inserted through the slot 26 in the top surface 22 of the device 2. When the cartridge 27 has been loaded, it can lock with the carousel 70 and can be rotated with it. The cartridge 27 has separate pots 82 containing all the reagents that are specific to the particular assay to be carried out by that cartridge. The reagents can be in any form, such as solid, dried or liquid form. The reagents, if solid or dried, can be hydrated during operation by aqueous substances stored in other pots in the carousel. In a PCR analysis, for example, the reagents may include one or more of the following, namely: nucleic acid modifying enzymes (including DNAses, RNAses and restriction endonucleases); PCR primers; PCR probes; polymerases; reverse transcriptases; dual-mode enzymes such as polymerase/reverse transcriptase; and antibodies. Other reagents are possible. A sleeve 85 at the lower end of the cartridge 27 can be shaped such that it is pushed up during insertion via the slot 26 to enable the cartridge to be loaded in the carousel 70. The pots 82 to 84 can be covered by a breakable cover seal 85, such as a foil seal, or the like and, optionally, by a protective cap 86, such as a molded cap. On its upper surface the cap 86 can carry a machine-readable identification, such as, for example, 2-D barcode 87 or some other form of machine-readable identification, such as an electronic memory chip or RFID. A machine-readable identification 87 on the cartridge 27 can be held up to a reader 88 adjacent the mounting bay 16 in which the preparation device 2 is inserted. The reader can be separate from the analysis device, such as, for example, a handheld readers. The analysis instrument 1 can recognize the machine-readable identification code, and based on information contained in the machine-readable identification code, instruct the module associated with that mounting bay 16 to drive the preparation device 2 to carry out the necessary steps associated with the sample and reagents. It also can instruct the analysis instrument 1 to make the appropriate PCR thermal cycling and data analysis operations for the particular substance being detected.

By supplying the specific reagents in a cartridge separately from the rest of the sample preparation device, the cost of providing sample preparation devices for different substances can be kept to a minimum. The user does not need to stock a range of different sample preparation devices for different substances but need only stock the different reagent cartridges and a smaller number of common sample preparation devices.

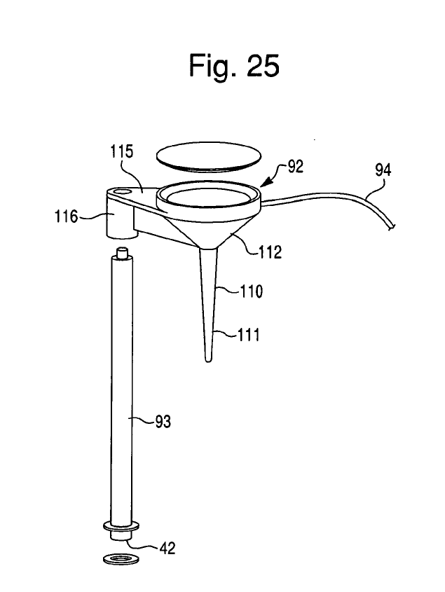

The mixture of the sample substance added via the inlet 25 can be exposed to the reagents and other treatment substances by means of, for example, a syringe pipette mechanism 90, shown most clearly in FIGS. 11, 12, 17, 18 and 25. The mechanism 90 can consist of two main parts: a syringe assembly 91 and a pipette assembly 92. The syringe assembly 91 can extend axially through the central aperture 71 in the carousel 70 and can be operable to effect pumping of fluid. The pipette assembly 92 can be mounted on a vertical elevator shaft 93 at the edge of the carousel and can be movable vertically up and down. The pipette assembly 92 and syringe assembly 91 can be connected via a length of flexible tubing 94. The syringe assembly 91 can have a central, axial worm shaft 95, which is externally threaded and, at is lower end provides the central drive coupling 40. The shaft 95 is fixed against axial displacement but is freely rotatable about its axis. Surrounding the shaft 95 is a cylindrical plunger 96, which can close at its upper end 97 and can be internally threaded in engagement with the external thread on the shaft 95. The syringe assembly 91 can be completed by an external hollow barrel 98, which can be fixed against axial or rotational movement. The upper end of the barrel 98 can be formed with a reduced diameter nose 99 to which one end of the tubing 94 is fixed. The upper end of the plunger 96 can support an O-ring 100, which makes a sliding seal with the inside surface of the barrel 98. The plunger 96 is shaped or provided with a surface formation to prevent it rotating relative to the barrel 98 so that, when the shaft 95 is rotated this is translated into axial displacement of the plunger along the inside of the barrel, so as to vary the volume of the chamber or potential space 101 at the upper end of the barrel. It can be seen, therefore, that rotation of the drive shaft 95 can be effective to cause pumping of air along the tubing 94.

The pipette assembly 92 can include a pipette 110 made of any suitable material and manufactured in any suitable way. In one embodiment, the pipette assembly 92 can be molded of a plastics material. The pipette 110 can have an elongate, tapering, vertically-oriented hollow stem 111 opening at its upper end into a closed conical receptacle 112. The receptacle 112 can have a small-bore spigot 113 projecting laterally generally towards the syringe assembly 91 and can receive the other end of the tubing 94. The internal volume of the stem 111 and receptacle 112 can be selected to be sufficient to contain any volume of liquid to be transferred by the pipette 90. In this way, it can be seen that operation of the syringe assembly 91 can be effective to pump air or gas above the liquid in the receptacle 112 and that no liquid need flow through the tubing 94 into the syringe chamber 101.

The pipette 110 can be supported by an arm 115, which extends laterally of the pipette and can be terminated by a threaded nut 116. The nut 116 can embrace the elevator shaft 93, which can be externally threaded and which can provide at its lower end the input drive coupling 42. It can be seen that the pipette 110 can be raised or lowered by appropriately rotating the elevator shaft 93 in different directions. In this way, both actuation of the syringe 91 and displacement of the pipette 110 can be accomplished by rotational drive inputs.

Fluid can be transferred between pots 75 in the carousel 70 by rotating the carousel so that the appropriate pot is positioned directly below the pipette 110; lowering the pipette into the pot (breaking through the seal 76 if this has not already been broken); displacing the syringe 91 to cause a reduced pressure in the pipette 110 and thereby suck up the fluid into the pipette; raising the pipette to allow the carousel to be rotated to position the desired pot directly beneath it; lowering the pipette into the pot; and then driving the syringe to increase gas pressure above the fluid in the pipette and force it out into the pot. Mixing within the pots can be promoted by repeatedly sucking and expelling fluid into the pipette 110 so as to cause flow of fluid within the pot. Each pot 75 can be shaped with a tapering V-shape floor provided by two planar inclined surfaces. The lowest point can be located centrally, in line with the stem 111 of the pipette 110 so that the tip of the pipette can be lowered into the lowest point to enable extraction of the maximum amount of fluid from the pots.

During various of the fluid transfer stages of the preparation device 2 it may be desirable to prevent contamination of certain fluids by other fluids that have previously been transferred by the pipette. Even dispensing the entire contents of the pipette would not guarantee complete removal of its contents since some fluid could remain clinging to the tip of the pipette. Some previous arrangements have overcome this problem by changing the tip of the pipette but this complicates operation of the apparatus. The risk of contamination can be reduced by arranging for the device to lower the tip of the notionally-empty pipette 110 onto the layer 77 of fluid-absorbing material on top of the carousel 70, whenever it is necessary to prevent transfer of fluid. Any fluid clinging to the tip of the pipette 110, either on its outside or inside can be wicked away from the pipette by the absorbent material 77 and remains trapped in the material. This provides a simple, low-cost arrangement for preventing undesirable fluid transfer. In some cases, however, the pipette can be replaced to avoid the risk of contamination, such as when the sample is potentially dangerous.

In an embodiment, paramagnetic beads can be used to capture nucleic acid in the sample, and the beads can be subsequently washed to remove unwanted substances whilst retaining the nucleic acid for subsequent treatment or release, according to methods well-known in the art. The beads can be stored in aqueous solution in the pot “M” of the carousel 70, for example. The beads can be washed in the usual way by using a magnet to draw the beads with the nucleic acid bound to them out of suspension to a location and retaining the beads there while unwanted material is removed. The magnet assembly 45 (FIG. 20) can comprise two permanent bar magnets 121 and 122. The magnets 121 and 122 can be mounted substantially parallel to one another and substantially vertically on a lateral, horizontal polepiece 123. The pole-piece 123 can be comprised of any suitable material, such as, for example, soft-iron. The two magnets can be oriented in opposite senses so that the north pole of one 121 and the south pole of the other 122 is uppermost. When not required, the magnet assembly 45 can be located at the far end of the slots 46 and 47 away from the pipette station. When magnetic separation is to be carried out, the analyzer 1 can displace a carriage 124 along a slot 125 in the upper surface of the mounting bay 16 to engage and displace the magnet assembly 45 along the slots 46 and 47 so that it moves to a position directly below the pipette 110. In this position, the two permanent magnets 121 and 122 are located on opposite sides of the carousel pot 75 directly below the pipette 110 (as shown in FIG. 20) so that the magnetic field set up by the magnet assembly 45 passes through the wall of the pot and into the fluid and magnetic bead suspension. The magnet assembly 45 can be arranged such that the magnetic field can be concentrated in two localized regions 127 and 128 on opposite sides of the tapered floor of the pot 75 and spaced above the lowest point or sump region 129. Magnetic beads are, therefore, attracted to these two regions 127 and 128, leaving the sump region 129 clear of beads so that the tip of the pipette 110 can be lowered into this region and the maximum volume of fluid extracted. The magnet assembly 45 is then moved back to its original position to allow the magnetic beads to move freely in the next fluid added to the pot 75.

As an example, the sample preparation device 2 can be arranged to carry out the following steps:
1. Acceptance of a sample into the device
2. Maceration and/or mixing of the sample with a volume of lysis/binding buffer to inactivate nucleases and release nucleic acids from the sample.
3. Mixing of the macerated sample with a further volume of lysis binding buffer, to release nucleic acid from the sample.
4. Mixing of the sample with a substance, such as proteinase K, to further break down the sample and release nucleic acids.
5. Capture of the nucleic acid so released onto paramagnetic beads, for example.
6. Washing of the nucleic acid (including the beads in some embodiments) with a defined buffer.
7. In the case where the target nucleic acid is RNA, optional incubation of the washed nucleic acid with a solution containing DNAseI.
8. In the case where the target nucleic acid is DNA, optional incubation of the so washed nucleic acid with a solution containing RNAse.
9. Further wash(es) of the nucleic acid with one or more defined buffers.
10. If beads were used, mixing of the beads bearing the target nucleic acids with a solution which elutes off the target nucleic acid.
11. Incubation of this eluate with a mixture of optionally freeze-dried nucleic acid primers and probes.
12. Incubation of the mixture formed in 11 with one or more freeze-dried DNA modifying enzymes or polymerases.
13. Transfer of the mixture formed in 12 to the PCR cuvette 30 via a cuvette filling mechanism.
14. Withdrawal of the cuvette filling mechanism from the cuvette 30.
15. Addition to the top of the cuvette a quantity of a material such as light mineral oil, or other materials to prevent subsequent evaporation of the mixture.

After these steps have been carried out the mixture within the cuvette 30 can be placed to carry out thermocycling to effect any suitable reaction, such as, for example, PCR, LATE-PCR, reverse transcriptase (RT)-PCR, within the analyzer 1.

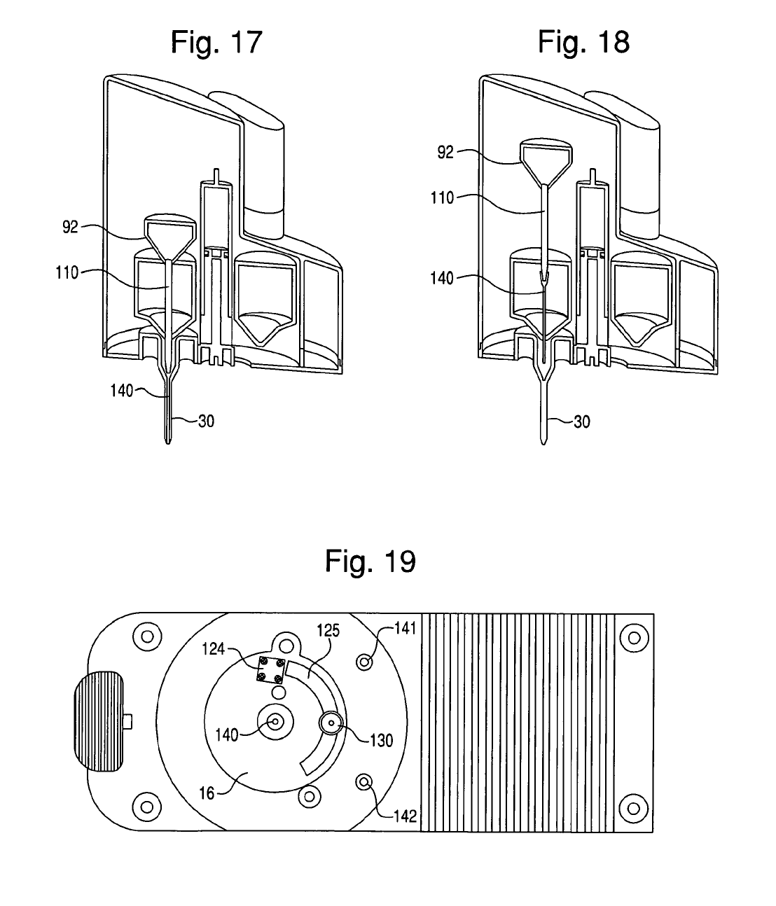

An exemplary cuvette filling mechanism is shown in more detail in FIGS. 9, 17, 18 and 24. In this example, the cuvette 30 can be of conventional form being molded from an optically-transparent plastics material suitable for use in the PCR reaction and optical detection steps in the analyzer 1. The cuvette 30 can be fixed in position vertically in an aperture 138 in the floor 28 of the housing 20 and does not move with the carousel 70. It is in alignment, directly below the pipette 110. When the cuvette 30 is to be filled, the pipette 110 can be loaded with fluid and the carousel 70 is rotated until a circular aperture 131 immediately to the left of the first pot “A” is located above the cuvette, so that the upper end of the cuvette is exposed for access by the pipette. The cuvette filling mechanism can comprise a plastics capillary tube 140, which is open at both ends and is located to extend within the cuvette 30 to contact the inside of its lower end 141. The upper end of the capillary 140 has a tapering coupling 142 fitted to it, which is shaped to engage with the outside of the tip of the pipette 110. The capillary 140 is a free sliding fit within the cuvette 30 allowing a gas venting clearance around it. To fill the cuvette 30, the pipette 110 can be lowered to engage with the coupling 142 on the capillary 140, as shown in FIG. 17, and the syringe 91 can be actuated slowly to pump out the fluid in the pipette. The fluid flows down the capillary 140, the clearance between the outside of the capillary and the inside of the cuvette 30 being sufficient to allow air to vent from the cuvette as it is filled with fluid. The fluid can flow out of the bottom end of the capillary 140 and can wick up into the annular clearance between the outside of the capillary and the inside of the cuvette. The pipette 110 can then be raised, taking with it the capillary tube 140, which can be attached by the coupling 142, as shown in FIG. 18. The next action, as mentioned in step 15 above, can be to index the carousel 70 one station clockwise so that a small oil reservoir 150 is located below the pipette 110. The capillary tube 140 attached to the end of the pipette 110 can be lowered into the oil reservoir 150 and its contents aspirated into the pipette. The carousel 70 can then be indexed back by one station anticlockwise and the capillary 140 can be lowered into the upper end of the cuvette 30 to dispense a small quantity of oil onto the top of the fluid in the cuvette 30 to prevent evaporation.

The analyzer 1 with which a sample preparation device is used could include provision to record the location of the analyzer so that this information can be stored with the results of analysis. The location information could be entered manually or via an internal or external GPS or similar positioning system. The analyzer also can be capable of transmitting the location of the analyzer to a remote location. For example, the analyzer can transmit its location to a remote location at periodic intervals, when prompted by the user, or whenever an analysis is performed.

Although the sample preparation device is particularly suited to preparation of biological samples for PCR analysis and related techniques, it could also be used for preparing other samples for analysis by different analyzers.

Where the prepared sample in the cuvette is of biological origin the reactions or transformations that take place during analysis may typically include any of the following: polymerase chain reaction (including variants thereof, including Linear After The Exponential (LATE) PCR); reverse transcription; exonuclease activity; endonuclease activity; and hybridisation or binding with other reagents such as oligonucleotides or antibodies. Other reactions and transformations are also possible.

The device described herein can be adapted for use in any location, such as in the field or in a stationary setting, such as, for example, a doctor's office, clinic or laboratory. The device described herein can allow an unskilled user to perform the sample preparation for PCR, and the PCR itself, in a small, self-contained, single-use consumable, which is controlled entirely by a field-portable instrument, and requires no knowledge of molecular biology. The device could be used in veterinary applications to prepare samples for detection of, for example. foot and mouth, avian flu and blue tongue or other diseases. The device enables rapid detection to be carried out in the region from where the samples are obtained so that rapid action can be taken if the a disease is detected. If a negative response is produced it avoids the need to take unnecessary, costly precautionary measures of the kind that would be necessary if the sample had to be sent to a remote location for laboratory analysis.

What is claimed is:

1. An apparatus for separating magnetic particles from a fluid comprising (a) a container for holding a fluid, (b) magnetic particles, and (c) a magnet movable between a first position where its magnetic field attracts the magnetic particles to a surface of the container and a second position where its magnetic field has substantially no effect on the magnetic particles.

2. The apparatus of claim 1, wherein the magnet is movable along curved path in a plane substantially at right angles to the axis of the container.

3. The apparatus of claim 1, wherein the magnetic particles have a tapering, closed lower end portion.

4. The apparatus of claim 1, wherein the magnet is selectively positionable relative to the container adjacent the container's lower end, and wherein the magnet comprises two magnet pole pieces aligned substantially parallel with the axis of the container and arranged to form regions for collection of the magnetic particles along the lower end portion of the container and displaced from the lower end of the end portion of the container.

5. An apparatus for dispensing a fluid into a cuvette comprising a capillary tube open at both ends and extending within the cuvette, wherein the lower end of the capillary tube is in contact with the inside of the closed end of the cuvette, and wherein the upper end of the capillary tube is exposed at the upper end of the cuvette, and wherein the dimensions of the capillary tube are such that when the fluid is applied to the upper end of the capillary tube it flows to its lower end expelling air in the cuvette between the outside of the capillary tube and the inside of the cuvette.

6. A sample preparation device comprising the apparatuses of claims 1 and 5.

7. A sample preparation device comprising: (a) a fluid-transfer mechanism arranged to transfer material between different containers in the device, wherein the mechanism comprises two components movable relative to one another to define a chamber of variable volume; and (b) a dispensing device connected with the chamber, wherein the dispensing device is displaceable such that it can be raised and lowered relative the containers, and wherein the displacement of the dispensing device and the two components relative to one another both being brought about by a rotary drive.

8. The device of claim 7, wherein the two components are a barrel and a piston.

9. The device of claim 7, wherein the rotary drive comprises an external unit to which the sample preparation device can be mounted and demounted.

10. A sample preparation device comprising a sample inlet comprising a macerator, wherein the macerator derives a fluid material from a liquid, solid, or semi-solid sample material.

11. The device of claim 10, wherein the macerator comprises at least one reservoir of a treatment fluid protected by a breakable seal, the treatment fluid being brought into contact with the sample material by manual operation of the macerator means.

12. The device of claim 10, wherein the macerator comprises a rotatable macerator knob that seals the inlet and is screw threaded relative to the sample inlet such that rotation of the knob displaces the knob down to push the sample down through the macerator.

13. A sample preparation device comprising: (a) a plurality of containers; (b) a transfer device that is arranged to be raised and lowered and moved laterally relative to the containers to transfer substances between containers; and (c) an absorbent material arranged to collect excess substance at the lower end of the transfer device.

14. The device of claim 13, wherein the absorbent material is preferably disposed on the upper surface of the containers and may be a fabric or non-woven material.

15. The device of claim 13, wherein the transfer device is adapted to be lowered such that its lower end contacts the absorbent material when it is necessary to remove excess fluid from the end of the transfer device.

16. An analyzer arrangement comprising:
(a) a sample preparation device comprising at least one container containing and a device for extracting fluid from the container, wherein the sample preparation device is mountable with and demountable from the base unit; and
(b) a base unit comprising a mechanism for moving a magnet between a first position and a second position, wherein the magnet can attract the contents of the container in the first position, and wherein the magnetic has substantially no effect on the contents of the container in the second position.

17. The analyzer of claim 16, wherein the magnet means is movable along an arc in a plane at right angles to the axis of the container and wherein the magnet is mounted on the sample preparation device.

18. An apparatus comprising:
(a) an analyzer; and
(b) a sample preparation device comprising a plurality of containers, wherein at least some of the containers are provided as a single component, wherein at least one of the containers is provided separately of the component and being mountable with the component, wherein the separately-provided container being provided with a machine-readable identification that can be read by the analyzer to control the analyzer to drive the sample preparation device in a particular sequence, and wherein the sample preparation device is mountable with and demountable from the analyzer.

19. A method of identifying a substance comprising:
(a) providing a sample preparation device having a plurality of installed containers of substances suitable for use in preparation of a range of different substances;
(b) providing a range of at least two separate containers containing different substances suitable for use in preparation of at least two respective different substances;
(c) selecting one of the separate containers according to the substance to be detected;
(d) installing the selected separate container with the sample preparation device;
(e) adding the sample substance to the sample preparation device;
(f) operating the sample preparation device to prepare the substance; and
(g) identifying the prepared substance.
